(19) United States
(12) Patent Application Publication
(10) Pub. No.: US 2009/0115997 A1
(43) Pub. Date: May 7, 2009

(54) MEASUREMENT SYSTEMS AND METHODS FOR OXYGENATED HEMOGLOBIN SATURATION LEVEL

(75) Inventors: Chih-Chung Yang, Taipei City (TW); Chih-Wei LU, Taipei City (TW); Cheng-Kuang Lee, Taipei City (TW); Meng-Tsan Tsai, Taipei City (TW); Yih-Ming Wang, Taipei City (TW)

Correspondence Address: QUINTERO LAW OFFICE, PC, 2210 MAIN STREET, SUITE 200, SANTA MONICA, CA, 90405, US

(73) Assignee: NATIONAL TAIWAN UNIVERSITY, TAIPEI (TW)

(21) Appl. No.: 12/031,397

(22) Filed: Feb. 14, 2008

(30) Foreign Application Priority Data
Nov. 1, 2007 (TW) TW96141175

(51) Int. Cl. G01N 33/48

(52) U.S. Cl. 356/41

(57) ABSTRACT

Measurement system and methods for measuring oxygenated hemoglobin saturation level are provided. Light is transmitted to test blood and a reference mirror. The reference mirror provides a first reflected light beam, and backscattered light from different depths of the test blood generates a second reflected light beam. An interfered light signal is generated by light interference of the first and second reflected light beams. According to the interfered light signal, a first light decay constant for a first light wavelength range and a second light decay constant for a second light wavelength range are obtained according to the interfered light signal. A decay ratio of the first light decay constant to the second light decay constant is obtained. Oxygenated hemoglobin saturation level of the test blood is obtained according to the decay ratio.

### CROSS REFERENCE TO RELATED APPLICATIONS

This application claims the benefit of Taiwan application Serial No. 96141175 filed Nov. 1, 2007, the subject matter of which is incorporated herein by reference.

### BACKGROUND OF THE INVENTION

1. Field of the Invention

The invention relates to a measurement method, and more particularly to a measurement system for measuring the oxygenated hemoglobin saturation level of blood.

2. Description of the Related Art

Oxygenated hemoglobin saturation level is an important factor for medical diagnoses. Some studies have provided a conclusion that cancerous tumor is related to the oxygenation level in blood. Since there are proliferous blood vessels near and around cancerous cells, tissue near and around the cancerous cells contains more oxygenated hemoglobin. Currently, several measurement devices are used to monitor the oxygenation level in blood, such as a blood gas analyzer. However, some of these devices measure the oxygenation level in blood by an invasive mode, and some take much time to measure oxygenation level in blood.

Thus, it is desired to provide a measurement method and device for measuring the oxygenated hemoglobin saturation level of blood rapidly and in a non-invasive mode.

### BRIEF SUMMARY OF THE INVENTION

An exemplary embodiment of a measurement method for measuring oxygenated hemoglobin saturation level is provided. First, test blood and a reference mirror are provided. Light is transmitted to the test blood and the reference mirror. The reference mirror provides a first reflected light beam, and backscattered light from different depths of the test blood generates a second reflected light beam. An interfered light signal is obtained by light interference of the first and second reflected light beams. A first light decay constant for a first light wavelength range and a second light decay constant for a second light wavelength range are obtained according to the interfered light signal. A decay ratio of the first light decay constant to the second light decay constant is calculated. Then, oxygenated hemoglobin saturation level of the test blood is obtained according to the decay ratio.

An exemplary embodiment of a measurement system for measuring oxygenated hemoglobin saturation level of a test blood is provided. The measurement system comprises a reference mirror, a spectroscope, a light source, a detection module, and a calculation module. The light source provides light to the reference mirror and the test blood through the light beam splitter. The reference mirror provides a first reflected light. The backscattered light from different depths of the test blood generates a second reflected light beam. Then, light interference occurs between the first and second reflected light beams. The detection module receives the interfered light signal generated by the light interference. The calculation module obtains a first light decay constant for a first light wavelength range and a second light decay constant for a second light wavelength range according to the interfered light signal. The calculation module calculates a decay ratio of the first light decay constant to the second light decay constant, and obtains oxygenated hemoglobin saturation level of the test blood according to the decay ratio.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows capabilities of oxygenated hemoglobin (HbO₂) and hemoglobin (Hb) absorbing light with different wavelengths;

FIG. 2 shows a spectroscopic spectral-domain optical coherence tomography (SSDOCT) device;

FIGS. 3a-1, 3b-1, 3c-1, 3d-1, 3e-1, and 3f-1 show intensity of short-wavelength light for the interference of the backscattered light from different depths of the blood sample 22 at 0, 2, 4, 6, 8, and 10 minutes after the blood sample is taken out from the oxygen container;

FIGS. 3a-2, 3b-2, 3c-2, 3d-2, 3e-2, and 3f-2 show intensity of long-wavelength light for the interference of the backscattered light from different depths of the blood sample 22 at 0, 2, 4, 6, 8, and 10 minutes after the blood sample is taken out from the oxygen container;

FIG. 4 shows the variation curves of μ_{long}/μₛₕₒᵣₜ and the oxygen pressure as time varies;

FIG. 5 shows an exemplary embodiment of a measurement system for measuring the oxygenated hemoglobin saturation level; and

FIG. 6 is a flow chart of an exemplary embodiment of a measurement method for measuring the oxygenated hemoglobin saturation level.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

FIG. 1 shows capabilities of oxygenated hemoglobin (HbO₂) and hemoglobin (Hb) absorbing light with different wavelengths, wherein a curve 10 represents the capability of HbO₂ absorbing light, and a curve 11 represents the capability of Hb absorbing light. Referring to the curves 10 and 11 in FIG. 1, for light with wavelength less than 800 nm, the absorption coefficient of HbO₂ is less (absorption capability is weaker), and the absorption coefficient of Hb is greater (absorption capability is stronger). For light with wavelength greater than 800 nm, the absorption coefficient of HbO₂ becomes greater (absorption capability becomes stronger), and the absorption coefficient of Hb becomes less (absorption capability becomes weaker). Accordingly, through the different characteristics of HbO₂ and Hb absorbing light with wavelength less than 800 nm and HbO₂ and Hb absorbing light with wavelength greater than 800 nm, the oxygenated hemoglobin saturation level in blood can be obtained.

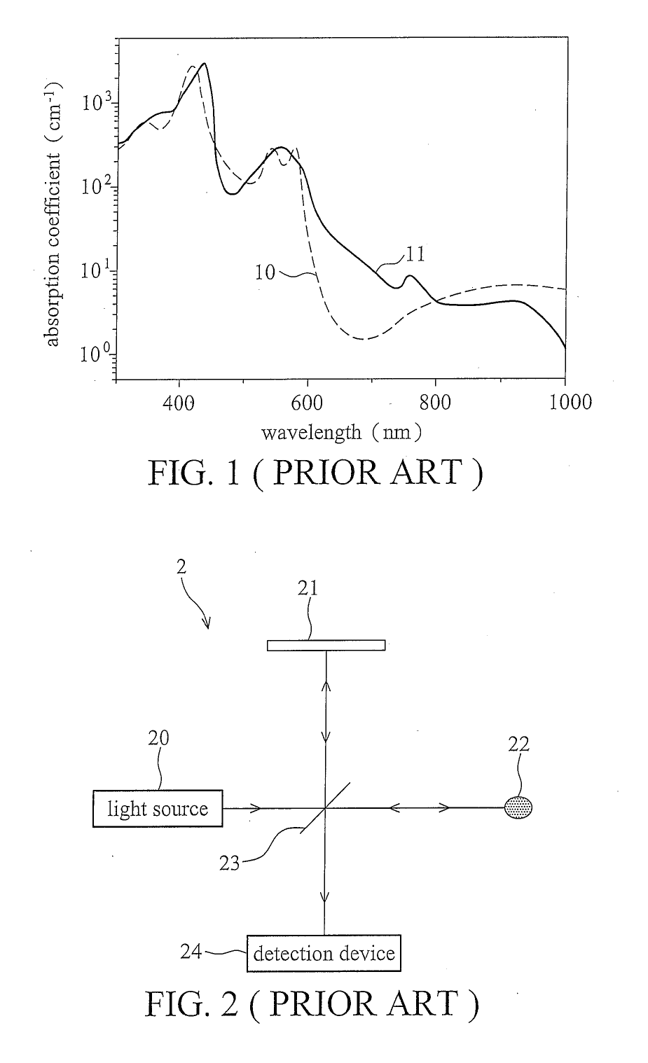

FIG. 2 shows a spectroscopic spectral-domain optical coherence tomography (SSDOCT) device 2. A light source 20 provides light with a central wavelength at 800 nm. A reference mirror 21 is disposed on one side of a spectroscope 23. A blood sample 22 is put on a culture dish, and the culture dish is placed in a container filled with pure oxygen for 30 minutes, so that Hb of the blood sample 22 is combined with oxygen. Then, the blood sample 22 is removed from the oxygen container and disposed on the other side of the light beam splitter 23. The light from the light source 20 is divided into two light beams through the light beam splitter 23. One light beam is transmitted to the reference mirror 21 and reflected by the reference mirror 21 to generate a first reflected light beam. The other light beam is transmitted to the blood sample 22, and part of the light beam is backscattered by suspended particles in different depths of the blood sample 22 to generate a second reflected light beam. The first and second reflected light beams meet through the light beam splitter 23, and light interference occurs between the first and second reflected light beams. A detector 24 receives the interference signal. The detector 24 comprises a spectrometer which measures intensity of light with different wavelengths in the interference signal. Through further calculation, intensity of backscattered signals from different depths of the blood sample 22 is obtained. The detector 24 provides the data for analyzing intensity decay characteristic of the backscattered signals from each depth in long-wavelength light and short-wavelength light demarcated by 800 m.

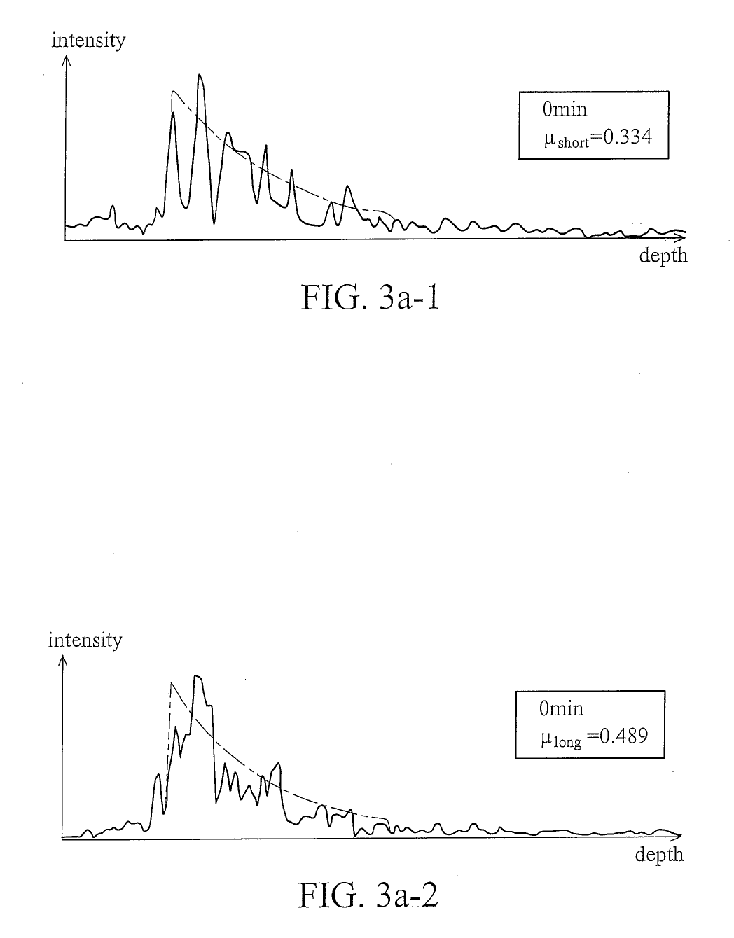

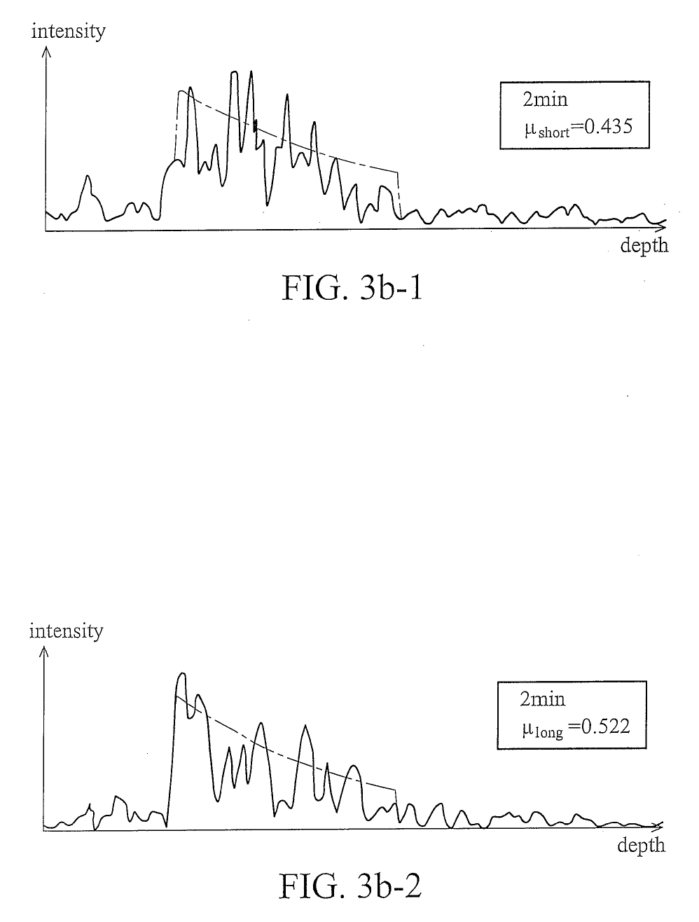

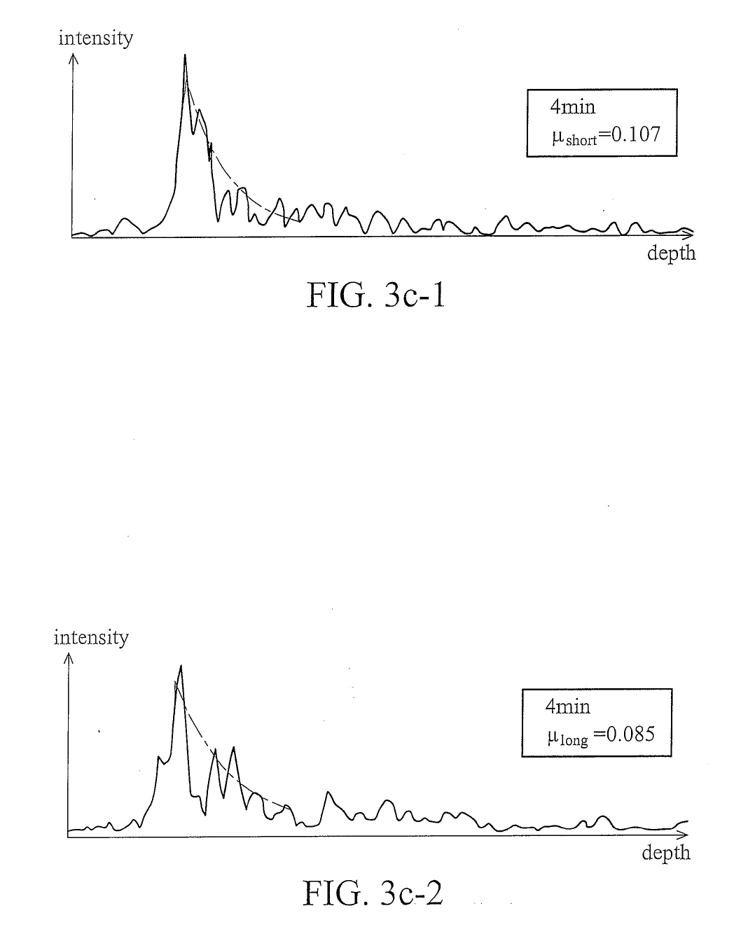

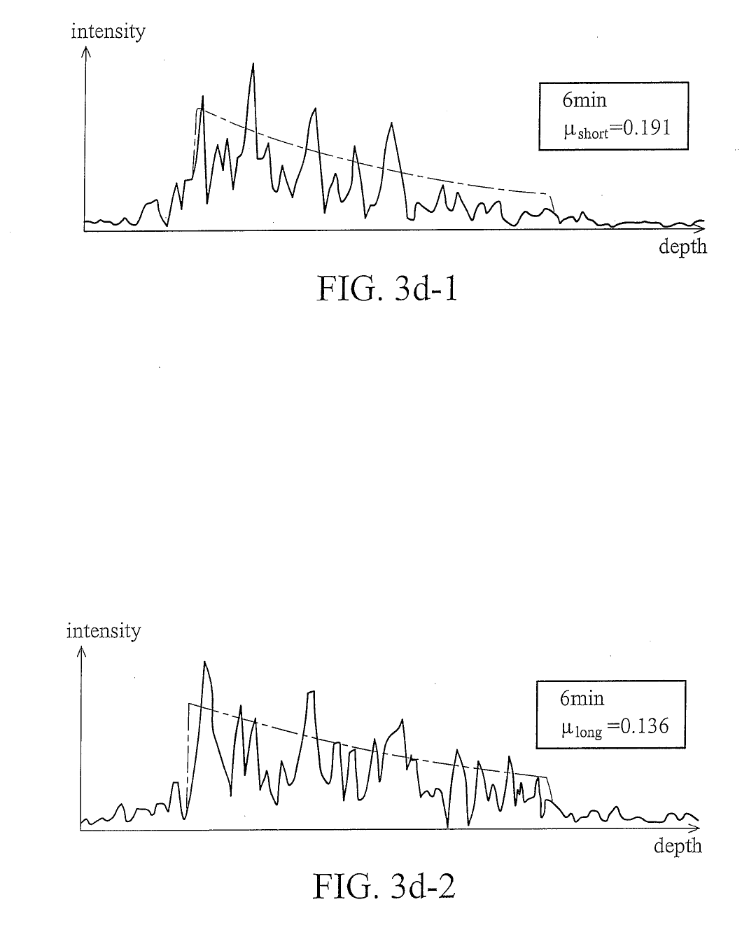

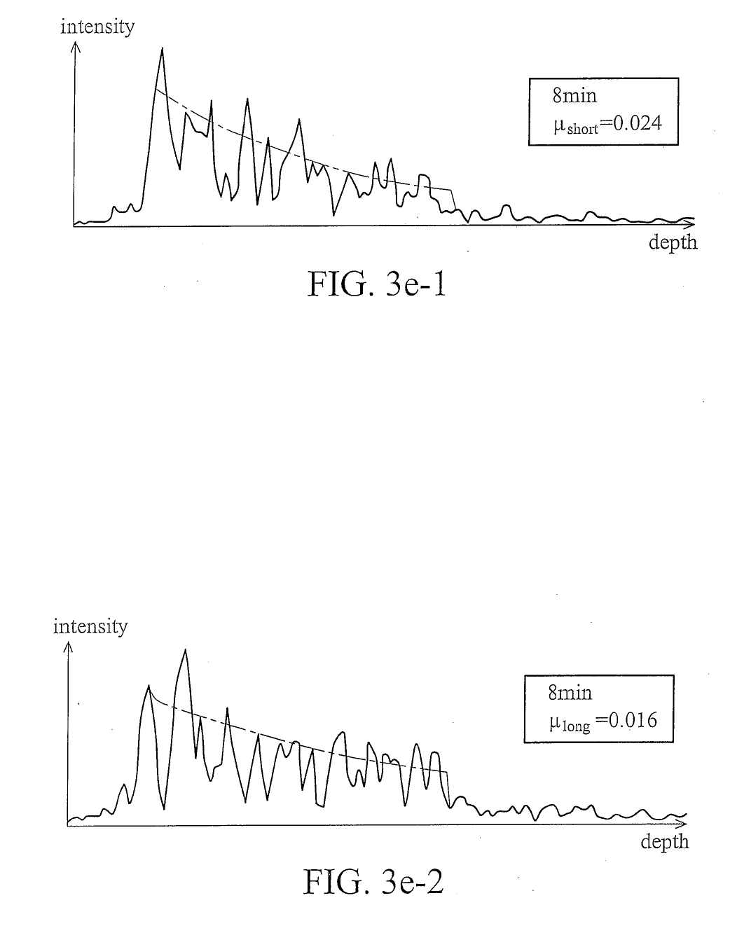

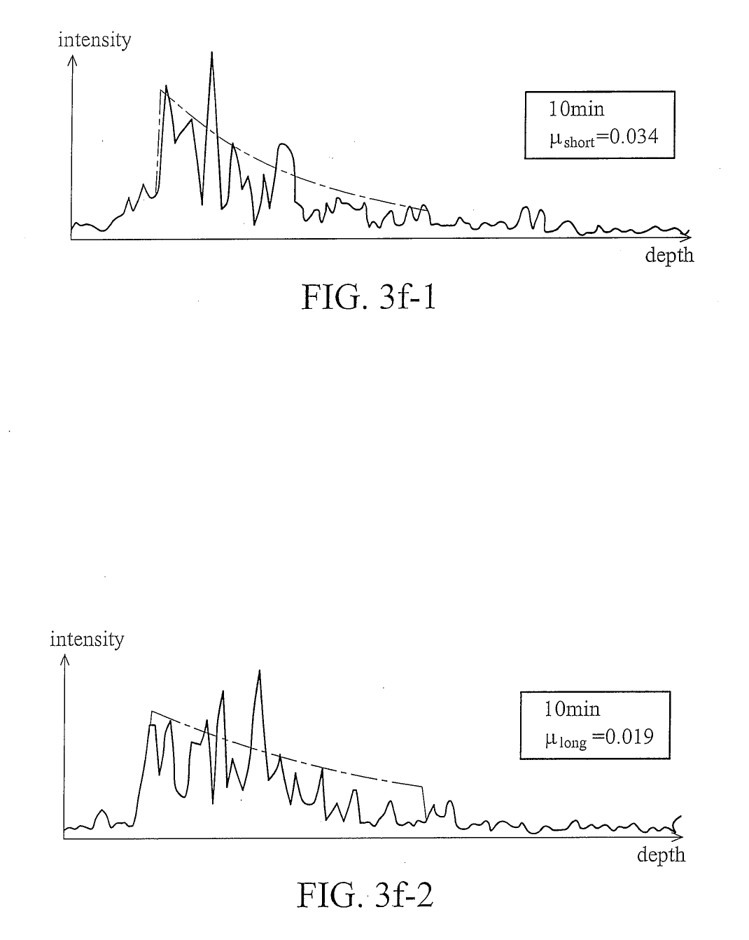

FIGS. 3a-1, 3b-1, 3c-1, 3d-1, 3e-1, and 3f-1 respectively show intensity of short-wavelength light for the interference of the backscattered light from different depths of the blood sample 22 at 0, 2, 4, 6, 8, and 10 minutes after the blood sample 22 is taken out from the oxygen container, wherein the horizontal axes represent depths of the blood sample 22, and the vertical axes represent light intensity. According to the intensity signals of short-wavelength light, light decay constants μₛₕₒᵣₜ are obtained. FIGS. 3a-2, 3b-2, 3c-2, 3d-2, 3e-2, and 3f-2 respectively show intensity of long-wavelength light for the interference of the backscattered light from different depths of the blood sample 22 at 0, 2, 4, 6, 8, and 10 minutes after the blood sample 22 is taken out from the oxygen container, wherein the horizontal axes represent depths of the blood sample 22, and the vertical axes represent light intensity. According to the intensity signals of long-wavelength light, light decay constants μ_{long} are obtained.

According to FIGS. 3a-1, 3a-2 . . . 3f-1, and 3f-2, when the blood sample 22 is taken out from the oxygen container in early stages (referred to FIGS. 3a-1, 3a-2, 3b-1, and 3b-2), the decay of long-wavelength light is faster than the decay of short-wavelength light (μ_{long}>μₛₕₒᵣₜ). That means that when the oxygen concentration of the blood sample 22 is high, the capability of the blood sample 22 absorbing long-wavelength light is stronger than the capability of the blood sample 22 absorbing short-wavelength light. Referring to FIG. 1, the characteristic of the capability of absorbing light when the blood sample 22 is taken out from the oxygen container in the early stages conforms with the characteristic of the capability of HbO₂ absorbing light with the long wavelength and the short wavelength. Referring to FIGS. 3c-1, 3c-2 . . . 3f-1, and 3f-2, as time goes by, the oxygen concentration in the blood sample 22 becomes less, and the decay of short-wavelength light is faster than the decay of long-wavelength light (μₛₕₒᵣₜ>μ_{long}). That means that when the oxygen concentration in the blood sample 22 is low, the capability of the blood sample 22 absorbing long-wavelength light becomes weaker than the capability of the blood sample 22 absorbing short-wavelength light. Referring to FIG. 1, the characteristic of the capability of absorbing light when the blood sample 22 is taken out from the oxygen container in the late stages conforms with the characteristic of the capability of Hb absorbing light with the long wavelength and the short wavelength. According to the variations of the light decay constants μ_{long} and μₛₕₒᵣₜ, the ratio of the light decay constant μ_{long} to the light decay constant μₛₕₒᵣₜ (μ_{long}/μₛₕₒᵣₜ) is related to the oxygenated hemoglobin saturation level.

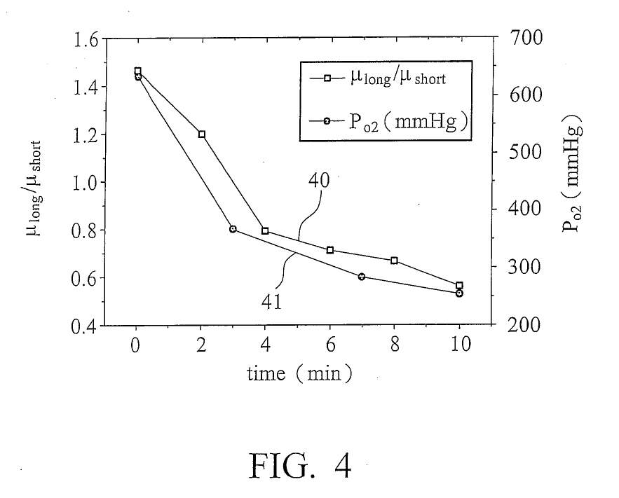

Moreover, another blood sample is taken to be placed in another oxygen container for 30 minutes, so that Hb of the blood sample is combined with oxygen. Then, the blood sample is removed from the oxygen container, and oxygen pressure of the blood sample is measured by a conventional blood gas analyzer to serve as a reference group. FIG. 4 shows the variation curves of μ_{long}/μₛₕₒᵣₜ and the oxygen pressure as time varies. In FIG. 4, the horizontal axis represents time, the left vertical axis represents values of μ_{long}/μₛₕₒᵣₜ, and the right vertical axis represents values of oxygen pressure (P_{O2}). The curve 40 for the blood sample 22 represents a variation curve of the values of μ_{long}/μₛₕₒᵣₜ as time varies and corresponds to the left axis. The curve 41 for the blood sample of the reference group represents a variation curve of the values of oxygen pressure (P_{O2}) as time varies and corresponds to the right axis. Referring to FIG. 4, the curve 40 approximates the curve 41. Thus, after the spectroscopic spectral-domain optical coherence tomography (SSDOCT) device 2 performs the above light interference, the decay of long-wavelength light μ_{long}, the decay of short-wavelength light μₛₕₒᵣₜ, and the ratio μ_{long}/μₛₕₒᵣₜ are calculated, and variation of oxygenated hemoglobin saturation level can be obtained according to the variation of the values of μ_{long}/μₛₕₒᵣₜ.

The present invention provides a measurement system and method for measuring the oxygenated hemoglobin saturation level by using the ratio μ_{long}/μₛₕₒᵣₜ.

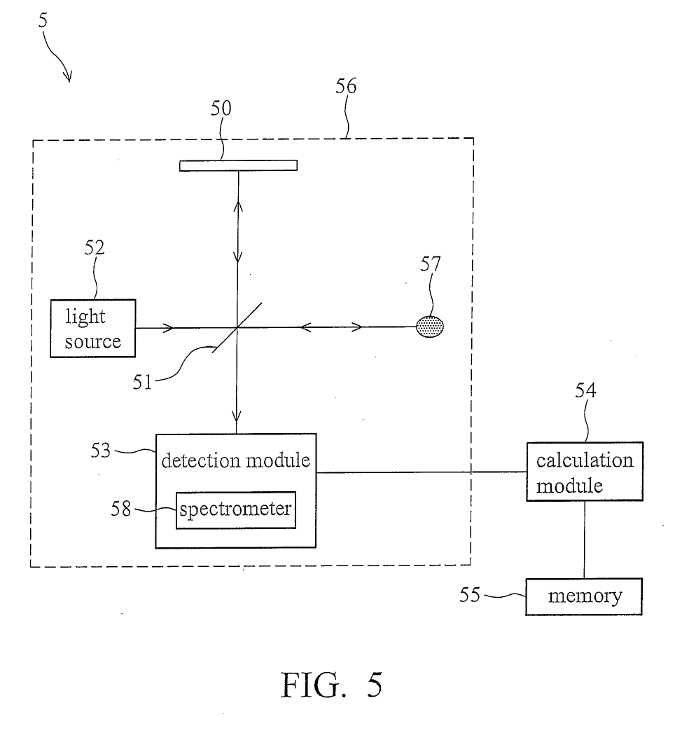

In an exemplary embodiment of a measurement system for measuring the oxygenated hemoglobin saturation level in FIG. 5, a measurement system 5 comprises a reference mirror 50, a light beam splitter 51, a light source 52, a detection module 53, a calculation module 54, and a memory 55. The reference mirror 50, the light beam splitter 51, the light source 52, and the detection module 53 make up an SSDOCT device 56. The detection module 53 comprises a spectrometer 58. The reference mirror 50 is disposed on one side of the light beam splitter 51, and the test blood 57 is disposed on the other side thereof. The light source 52 provides light. The light is divided into two light beams. One light beam is transmitted to the reference mirror 50 and reflected by the reference mirror 50 to generate a first reflected light beam. The other light beam is transmitted to the test blood 57, and the backscattered light by suspended particles of the test blood 57 generates a second reflected light beam. The first and second reflected light beams meet through the light beam splitter 51, and light interference occurs between the first and second reflected light beams.

The detection module 53 receives an interfered light signal generated by the light interference of the first and second reflected light beams. The spectrometer 58 of the detection module 53 measures a spectrum of the interfered light signal as following description. The spectrometer 58 measures intensity of the interfered light signal in different wavelengths. In this embodiment, the detection module 53 divides the full wavelength of the interfered light signal into a long-wavelength range and a short-wavelength range by 800 nm. The long-wavelength range greater than 800 nm is referred to as the first wavelength range, and the short-wavelength range less than 800 nm is referred to as the second wavelength range.

According to the intensity of the interfered light signal in the long-wavelength range and the short-wavelength range, the calculation module 54 obtains the decay characteristic of backscattered light from different depths of the test blood 57 in this two ranges, and further obtains a first light decay constant μ_{long} and a second light decay constant μₛₕₒᵣₜ. The calculation module 54 calculates a decay ratio of the light decay constant μ_{long} to the light decay constant μₛₕₒᵣₜ (μ_{long}/μₛₕₒᵣₜ). The memory 55 comprises a table recording values of oxygenated hemoglobin saturation level corresponding to different values of the decay ratio. After calculating the decay ratio, the calculation module 54 obtains the oxygenated hemoglobin saturation level of the test blood 57 by looking up the table according to the calculated decay ratio.

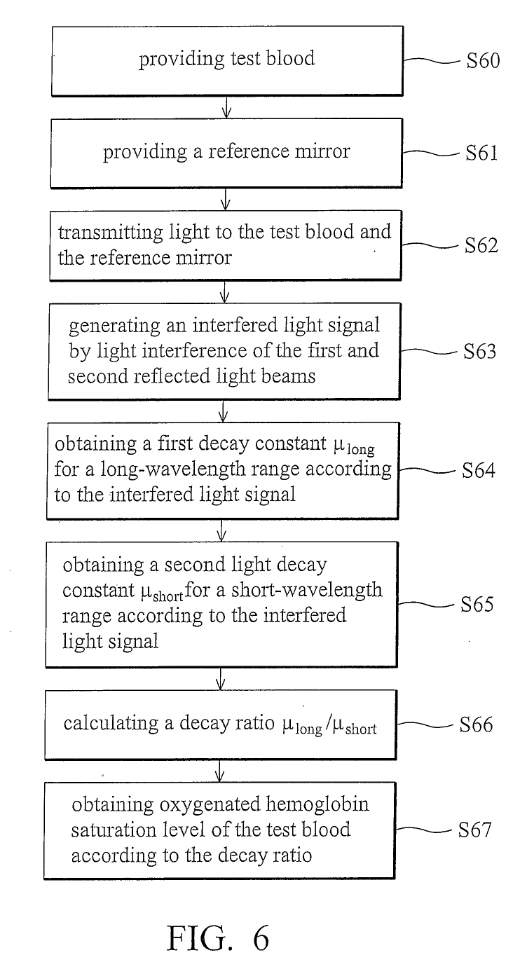

FIG. 6 is a flow chart of an exemplary embodiment of a measurement method for measuring the oxygenated hemoglobin saturation level. Referring to FIG. 6, first, test blood is provided (step S61), and a reference mirror is provided (step S62). Light is transmitted to the test blood and the reference mirror (step S62). In this embodiment, a reference mirror and a light source of an SSDOCT device is used to perform the steps S60-S62, such as the SSDOCT device 56 in FIG. 5 which comprises the reference mirror 50, the light beam splitter 51, the light source 52, and the detection module 53. The reference mirror 50 is disposed on one side of the light beam splitter 51, and the test blood is disposed on the other side of the light beam splitter 51, such as the position of the test blood 57. The light source 52 provides light to the test blood through the light beam splitter 51 and the reference mirror 50.

Light is reflected by the reference mirror 50 to generate a first reflected light beam, and backscattered light from different depths of the test blood 57 generates a second reflected light beam. The first and second reflected light beams meet through the light beam splitter 51, and a light interference is preformed to the first and second reflected light beams. Then, an interfered light signal is obtained by the light interference of the first and second reflected light beams (step S63). In the step S63 of the embodiment, the detection module 53 of the SSDOCT 56 is used to perform the step S63. The spectrometer 58 of the detection module 53 measures intensity of the interfered light signal in different wavelengths. The detection module 53 divides the full wavelength of the interfered light signal into a long-wavelength range and a short-wavelength range by 800 nm. The long-wavelength range greater than 800 nm is referred to as a first wavelength range, and the short-wavelength range less than 800 nm is referred to as a second wavelength range. Thus, according to the intensity distribution of the interfered light signal in the long-wavelength range and the short-wavelength range, the intensities of the backscattered light from different depths of the test blood are obtained, and light decay constants representing decay characteristic are further obtained.

A first light decay constant of the first wavelength range is obtained according to the interfered light signal (step S64), and a second light decay constant of the second wavelength range is obtained according to the interfered light signal (step S64). The decay ratio of the light decay constant μ_{long} to the light decay constant μₛₕₒᵣₜ (μ_{long}/μₛₕₒᵣₜ) is calculated (step S66). In the embodiment, the calculation module 54 is used to perform the steps S64-S66. After calculating the decay ratio (μ_{long}/μₛₕₒᵣₜ), the calculation module 54 obtains the oxygenated hemoglobin saturation level of the test blood 57 according to the calculated decay ratio by looking up a table, which records values of the oxygenated hemoglobin saturation level corresponding to different values of the decay ratio (step S67). In this embodiment, the table is stored in a memory, such as the memory 55 in FIG. 5.

In the embodiments above, the test blood can be a sample retrieved from a living body or blood in a living body. In other words, when the test blood is the blood in a living body, the measurement system measures the oxygenated hemoglobin saturation level of the blood in a non-invasive mode.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. A measurement method for measuring oxygenated hemoglobin saturation level, comprising:
providing test blood;
providing a reference mirror;
transmitting light to the test blood and the reference mirror, wherein the reference mirror provides a first reflected light beam, and backscattered light from different depths of the test blood generates a second reflected light beam;
generating an interfered light signal by light interference of the first and second reflected light beams;
obtaining a first light decay constant for a first light wavelength range according to the interfered light signal;
obtaining a second light decay constant for a second light wavelength range according to the interfered light signal;
calculating a decay ratio of the first light decay constant to the second light decay constant; and
obtaining oxygenated hemoglobin saturation level of the test blood according to the decay ratio.

2. The measurement method as claimed in claim 1, wherein the step of obtaining the interfered light signals comprises measuring a spectrum of the interfered light signal by a spectrometer.

3. The measurement method as claimed in claim 1, wherein the first light wavelength range includes wavelengths greater than 800 nm.

4. The measurement method as claimed in claim 1, wherein the second light wavelength range includes wavelengths less than 800 nm.

5. The measurement method as claimed in claim 1, wherein the light interference of the first and second reflected light beams is performed by a spectroscopic spectral-domain optical coherence tomography (SSDOCT) device for obtaining the interfered light signal.

6. The measurement method as claimed in claim 1 further comprising:
looking up a table according to the decay ratio for obtaining the oxygenated hemoglobin saturation level of the test blood.

7. The measurement method as claimed in claim 6, wherein the table is stored in a memory and records values of the oxygenated hemoglobin saturation level corresponding to different values of the decay ratio.

8. The measurement method as claimed in claim 1, wherein the test blood is a sample retrieved from a living body.

9. The measurement method as claimed in claim 1, wherein the test blood is blood in a living body.

10. A measurement system for measuring oxygenated hemoglobin saturation level of test blood, comprising:
a reference mirror;
a light beam splitter;
a light source providing light to the reference mirror and the test blood through the light beam splitter, wherein the reference mirror provides a first reflected light, backscattered light from different depths of the test blood generates a second reflected light beam, and a light interference occurs between the first and second reflected light beams;
a detection module receiving an interfered light signal generated by the light interference; and
a calculation module obtaining a first light decay constant for a first light wavelength range and a second light decay constant for a second light wavelength range according to the interfered light signal, calculating a decay ratio of the first light decay constant to the second light decay constant, and obtaining oxygenated hemoglobin saturation level of the test blood according to the decay ratio.

11. The measurement method as claimed in claim 10, wherein the detection module comprises a spectrometer measuring a spectrum of the interfered light signal, and the calculation module obtains the first and second light decay constants according to the spectrum of the interfered light signal.

12. The measurement method as claimed in claim 10, wherein the first light wavelength range includes wavelengths greater than 800 nm.

13. The measurement method as claimed in claim 10, wherein the second light wavelength range includes wavelengths less than 800 nm.

14. The measurement method as claimed in claim 10 further comprising a memory having a table, and the table recording values of the oxygenated hemoglobin saturation level corresponding to different values of the decay ratio.

15. The measurement method as claimed in claim 14, wherein the calculation module obtains the oxygenated hemoglobin saturation level of the test blood by looking up the table according to the decay ratio.
